(19) United States
(12) Patent Application Publication
(10) Pub. No.: US 2024/0268689 A1
(43) Pub. Date: Aug. 15, 2024

(54) PHYSIOLOGICAL SIGNAL MEASUREMENT APPARATUS

(71) Applicant: AUO Corporation, Hsinchu (TW)

(72) Inventors: Yuan Tsai Lin, Hsinchu (TW); Wen Ching Kao, Hsinchu (TW); Jung-Teng Pan, Hsinchu (TW); Chin-Tang Chuang, Hsinchu (TW)

(73) Assignee: AUO Corporation, Hsinchu (TW)

(21) Appl. No.: 18/460,580

(22) Filed: Sep. 3, 2023

(30) Foreign Application Priority Data
Feb. 13, 2023 (TW) 112105058

(51) Int. Cl. A61B 5/022

(52) U.S. Cl. CPC A61B 5/02233; A61B 2562/0247

(57) ABSTRACT

A physiological signal measurement apparatus, including a base seat, an air-bladder device, a plurality of pressure sensors, and a plurality of elastomers, is provided. The air-bladder device is disposed on the base seat and includes a plurality of sub air-bladders. The pressure sensors are respectively disposed on the sub air-bladders. The elastomers are respectively disposed between the sub air-bladders and the pressure sensors.

### CROSS-REFERENCE TO RELATED APPLICATION

This application claims the priority benefit of Taiwan application serial no. 112105058, filed on Feb. 13, 2023. The entirety of the above-mentioned patent application is hereby incorporated by reference herein and made a part of this specification.

### BACKGROUND

### Technical Field

The invention relates to a pressure measurement apparatus, and in particular to a physiological signal measurement apparatus.

### Description of Related Art

In the pulse diagnosis of traditional Chinese medicine, the doctor puts three fingers on the cunkou pulse of the patient and apply pressure to sense the changes in the pulse condition, and then combines all the information to complete the diagnosis. However, the pressing pressure of feeling the pulse and the judgment between different pulse conditions are mostly based on the personal diagnosis and treatment experience of the doctor to make a diagnosis. Since different doctors often have different standards on the pressing pressure, an objective and quantifiable pulse-diagnosing instrument is needed. However, current pulse diagnosis instruments cannot respectively apply appropriate pressures to chi, guan, and can with different depths, resulting in insufficient measurement accuracy.

### SUMMARY

The invention provides a physiological signal measurement apparatus, which can respectively apply appropriate pressures to pulse points with different depths to greatly improve the measurement sensitivity.

According to an embodiment of the invention, a physiological signal measurement apparatus including a base seat, an air-bladder device, a plurality of pressure sensors, and a plurality of elastomers is provided. The air-bladder device is disposed on the base seat and includes a plurality of sub air-bladders. The plurality of pressure sensors are respectively disposed on the plurality of sub air-bladders. The plurality of elastomers are respectively disposed between each of the plurality of sub air-bladders and a corresponding one of the plurality of pressure sensors.

Based on the above, by configuring the discrete sub air-bladders and elastomers, the physiological signal measurement apparatus provided by the embodiment of the invention can respectively apply appropriate pressures to pulse points with different depths, and each pressure sensor can correctly sense the pulse pressure at each pulse point to greatly improve the measurement sensitivity and shorten the measurement time.

In order to make the above-mentioned features and advantages of the invention more comprehensible, the following specific embodiments are described in detail together with the accompanying drawings.

### BRIEF DESCRIPTION OF THE DRAWINGS

FIG. 1A depicts a schematic view of a physiological signal measurement apparatus when worn on a hand of a user according to an embodiment of the invention. FIG. 1B and FIG. 1C depict cross-sectional views of the physiological signal measurement apparatus in FIG. 1A when worn on the hand of the user.

FIG. 2A and FIG. 2B depict partial schematic views of the physiological signal measurement apparatus according to an embodiment of the invention.

FIG. 3 depicts a schematic view of the physiological signal measurement apparatus according to an embodiment of the invention.

FIG. 4A depicts a pulse pressure curve diagram of a physiological signal measurement apparatus according to a comparative example. FIG. 4B depicts a pulse pressure curve diagram of the physiological signal measurement apparatus according to an embodiment of the invention.

FIG. 5A and FIG. 5B depict pulse pressure curve diagrams of the physiological signal measurement apparatus according to an embodiment of the invention.

FIG. 6A and FIG. 6B depict pulse wave signal diagrams of the physiological signal measurement apparatus according to an embodiment of the invention.

### DESCRIPTION OF THE EMBODIMENTS

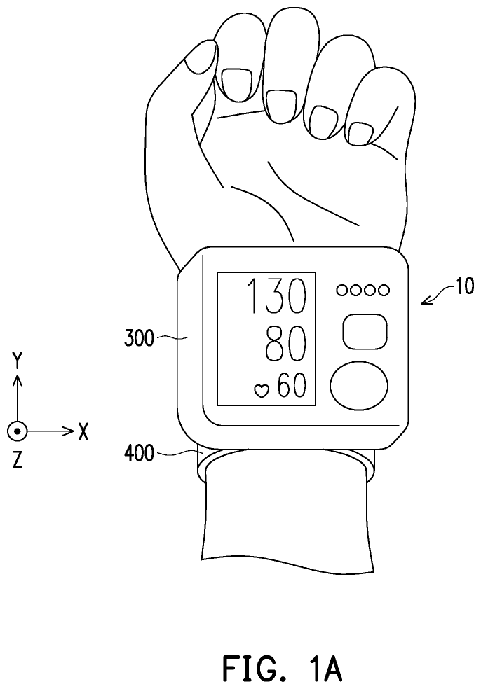

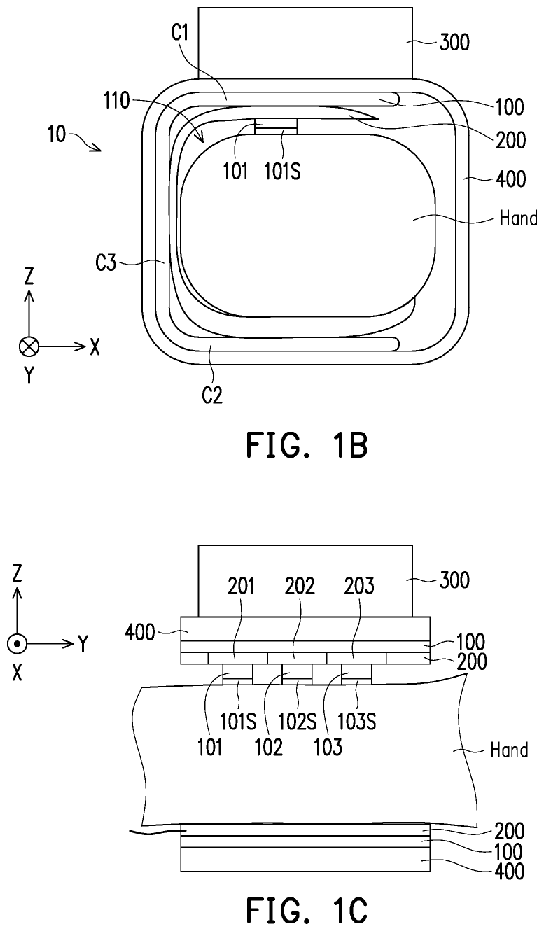

Referring to FIG. 1A to FIG. 1C, FIG. 1A depicts a top schematic view of a physiological signal measurement apparatus when worn on a hand of a user according to an embodiment of the invention. FIG. 1B and FIG. 1C depict cross-sectional views of the physiological signal measurement apparatus in FIG. 1A when worn on the hand of the user.

A physiological signal measurement apparatus 10 includes a base seat 100, an air-bladder device 200, a driving system 300, and a restraint 400. The base seat 100 includes a first wing C1 and a second wing C2 oppositely disposed and a connecting part C3 connecting the first wing C1 and the second wing C2, and the first wing C1, the second wing C2, and the connecting part C3 surround to form a containing part 110. The air-bladder device 200 is disposed in the containing part 110 to limit the expansion range of the air-bladder device 200 during the subsequently described process of inflating the air-bladder device 200.

In this embodiment, the first wing C1, the second wing C2, and the connecting part C3 of the base seat 100 are integrally formed to enhance the structural strength, and the first wing C1 and the second wing C2 are disposed in parallel to suit the hand placement, but the invention is not limited thereto.

The physiological signal measurement apparatus 10 also includes pressure sensors 101S, 102S, and 103S driven by the driving system 300 and elastomers 101, 102, and 103. The pressure sensor 101S is disposed on the elastomer 101, the pressure sensor 102S is disposed on the elastomer 102, and the pressure sensor 103S is disposed on the elastomer 103.

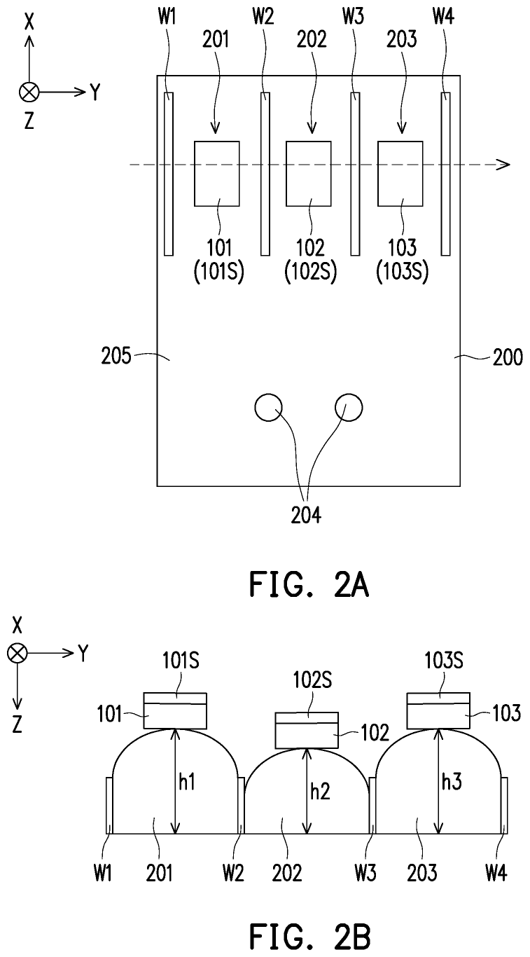

Please refer to FIG. 2A and FIG. 2B first. The air-bladder device 200 includes sub air-bladders 201, 202, and 203. The pressure sensor 101S is disposed on the sub air-bladder 201, and the elastomer 101 is disposed between the sub air-bladder 201 and the pressure sensor 101S. The pressure sensor 102S is disposed on the sub air-bladder 202, and the elastomer 102 is disposed between the sub air-bladder 202 and the pressure sensor 102S. The pressure sensor 103S is disposed on the sub air-bladder 203, and the elastomer 103 is disposed between the sub air-bladder 203 and the pressure sensor 103S.

The air-bladder device 200 further includes an inflation and deflation port 204, a communication area 205, and spacers W1, W2, W3, and W4. The number of the inflation and deflation port 204 may be one or more. The spacer W2 is disposed between the sub air-bladder 201 and the sub air-bladder 202, the spacer W3 is disposed between the sub air-bladder 202 and the sub air-bladder 203, the spacer W1 is disposed on a side of the sub air-bladder 201 away from the spacer W2, and the spacer W4 is disposed on a side of the sub air-bladder 203 away from the spacer W3. When gas is inflated into the air-bladder device 200 through the inflation and deflation port 204, due to the communication between the communication area 205 and the sub air-bladders 201, 202, and 203, the gas enters the sub air-bladders 201, 202, and 203 respectively after passing through the communication area 205, so that the sub air-bladders 201, 202, and 203 are in an inflated state as shown in FIG. 2B.

Please refer to FIG. 1B, FIG. 1C, and FIG. 2B at the same time. Since the air-bladder device 200 and the pressure sensors 101S, 102S, and 103S and the elastomers 101, 102, and 103 thereon are disposed in the containing part 110, the sub air-bladders 201, 202, and 203 are in the inflated state when the driving system 300 inflates the gas into the air-bladder device 200 through the inflation and deflation port 204. The expansion range of the air-bladder device 200 is limited by the base seat 100, so the inflated air-bladder device 200 can apply pressure on the hand of the user.

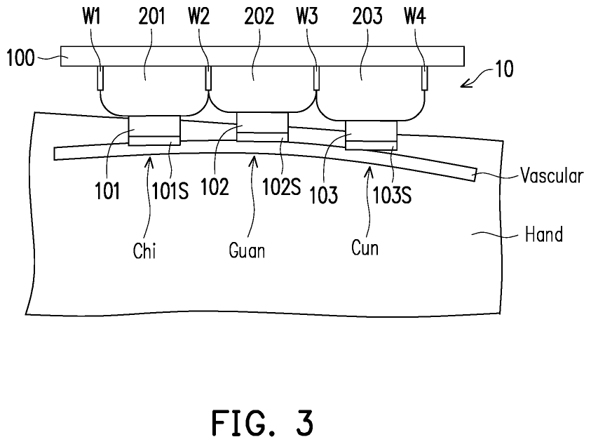

It should be noted that in the physiological signal measurement apparatus 10 of the embodiment of the invention, the pressure sensors 101S, 102S, and 103S are respectively disposed on the discrete elastomers 101, 102, and 103 and the discrete sub air-bladders 201, 202, and 203, so that the pressure sensors 101S, 102S, and 103S may be respectively attached to three points in contact with the hand, and the pressure can be concentrated on the three points without spreading to other positions in order to achieve sensitive measurement. Specifically, please refer to FIG. 1B, FIG. 2B, and FIG. 3 at the same time. In some embodiments, the physiological signal measurement apparatus 10 may be implemented as a pulse pressure measurement apparatus and is worn on the hand of the user through the restraint 400 (e.g. an elastic strap) to measure the pulse pressure of the cunkou pulse as shown in FIG. 3. The pressure sensor 101S is used to measure “chi”, the pressure sensor 102S is used to measure “guan”, and the pressure sensor 103S is used to measure “cun”, but the physiological signal apparatus 10 of the invention is not limited to measuring the cunkou pulse.

It should also be particularly noted that since the pulse points corresponding to the pressure sensors 101S, 102S, and 103S may be at different depth positions, the sub air-bladders 201, 202, and 203 are configured such that when the sub air-bladders 201, 202, and 203 are under the same inflation pressure, the heights of the sub air-bladders 201, 202, and 203 relative to the base seat 100 may be different. When the pulse point is deeper, the height of the corresponding sub air-bladder 201, 202, or 203 is higher, so as to ensure that each pulse point is applied with an appropriate pressure, so that the pressure sensors 101S, 102S, and 103S can all be attached to the hand to accurately sense the pulse pressure. Taking the measurement of the cunkou pulse shown in FIG. 3 as an example, since chi and the can are located deeper in the hand than guan, the heights of the sub air-bladder 201 and the sub air-bladder 203 relative to the base seat 100 are configured to be greater than the height of the sub air-bladder 202 relative to the base seat 100, so as to ensure that the pressure sensors 101S, 102S, and 103S can all sense the pulse pressure correctly. In an embodiment, heights h1 and h3 of the sub air-bladder 201 and the sub air-bladder 203 relative to the base seat 100 are 1.1 to 1.5 times a height h2 of the sub air-bladder 202 relative to the base seat 100. Moreover, attachment and sensitive measurement can be achieved by inflating through the single inflation and deflation port 204. However, the invention is not limited to the above-mentioned height ratio.

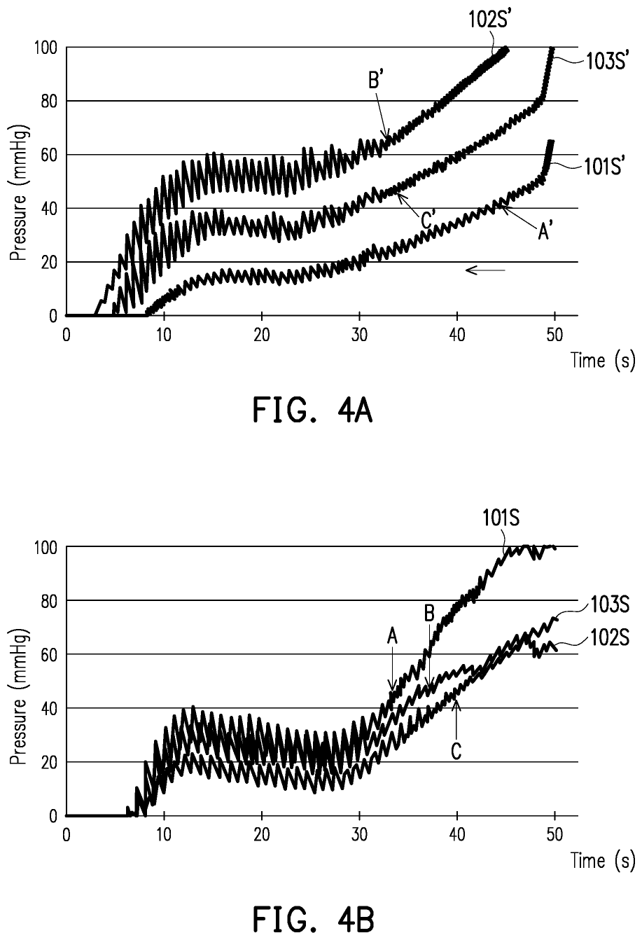

Referring to FIG. 4A and FIG. 4B, FIG. 4A depicts a pulse pressure curve diagram of the physiological signal measurement apparatus according to a comparative example. FIG. 4B depicts a pulse pressure curve diagram of the physiological signal measurement apparatus according to an embodiment of the invention. The horizontal axis is time, and the vertical axis is the air pressure in the sub air-bladder.

In the comparative example of FIG. 4A, the physiological signal measurement apparatus (not depicted) includes pressure sensors 101S′, 102 S′, and 103S′. The difference between the physiological signal measurement apparatus (not depicted) and the physiological signal measurement apparatus 10 of the invention is that sub air-bladders corresponding to the pressure sensors 101S′, 102S′, and 103S′ have the same height. When the physiological signal measurement apparatus of this comparative example measures the pulse pressure of the cunkou pulse, the pressure sensors 101S′, 102S′, and 103S′ respectively obtain sufficient pulse pressure data at points A′, B′, and C′ on the pulse pressure curve and end the measurement. It can be seen that in the measurement curve of the pressure sensor 101S′, the amplitude of each pulse wave is smaller, so the pressure sensor 101S′ needs a longer sensing time to collect enough pulse pressure data. As a result, the maximum interval between three time points corresponding to the points A′, B′ and C′ reaches 12 seconds, which lengthens the overall measurement time of the physiological signal measurement apparatus.

Next, please refer to FIG. 4B. Since the pressure sensors 101S, 102S, and 103S of the physiological signal measurement apparatus 10 of the invention are disposed on the discrete sub air-bladders 201, 202, and 203, and the sub air-bladders 201, 202, and 203 have different heights corresponding to the depths of chi, guan, and cun, appropriate pressures may be respectively applied to chi, guan, and cun, and the pressure sensors 101S, 102S, and 103S can respectively sense the pulse pressures of chi, guan, and cun without lengthening the sensing time due to unclear or insufficient pulse pressure data. Therefore, when the pressure sensors 101S, 102S, and 103S respectively obtain sufficient pulse pressure data at points A, B, and C on the pulse pressure curve and end the measurement, the maximum interval between three time points corresponding to the points A, B, and C is only about 7 seconds. Compared with the comparative example shown in FIG. 4A, the overall measurement time of the physiological signal measurement apparatus is greatly shortened.

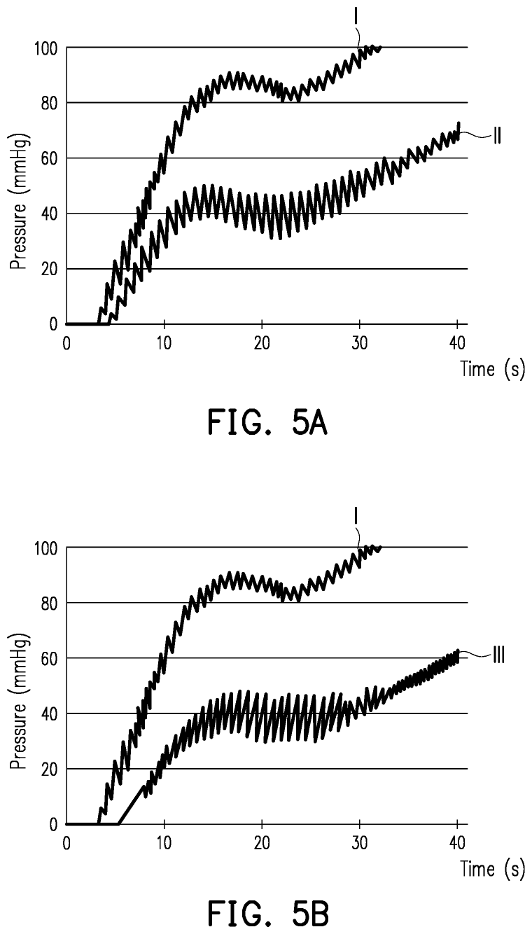

Referring to FIG. 3, FIG. 5A, and FIG. 5B at the same time, FIG. 5A and FIG. 5B depict pulse pressure curve diagrams of the physiological signal measurement apparatus according to an embodiment of the invention. Curve I represents the condition of the physiological signal measurement apparatus 10 not configured with the elastomers 101, 102, and 103. Curve II represents that the physiological signal measurement apparatus 10 is configured with the elastomers 101, 102, and 103, and each elastomer has a thickness of 2.5 mm and a hardness of 50 HA. Curve III represents that the physiological signal measurement apparatus 10 is configured with the elastomers 101, 102, and 103, and each elastomer has a thickness of 4.5 mm and a hardness of 10 HA.

As shown in FIG. 5A, when the physiological signal measurement apparatus 10 is configured with the elastomers 101, 102, and 103, and each elastomer has the thickness of 2.5 mm and the hardness of 50 HA (i.e. Curve II), the amplitude of each pulse wave is larger than that of the case without elastomers (Curve I), and the maximum amplitude can reach 15.32 mmHg. In contrast, the maximum amplitude of Curve I is only 12.88 mmHg. Similarly, as shown in FIG. 5B, when the physiological signal measurement apparatus 10 is configured with the elastomers 101, 102, and 103, and each elastomer has the thickness of 4.5 mm and the hardness of 10 HA (i.e. Curve III), the amplitude of each pulse wave is larger than that of the case without elastomers (Curve I), and the maximum amplitude can reach 15.76 mmHg, which is obviously greater than the maximum amplitude of Curve I.

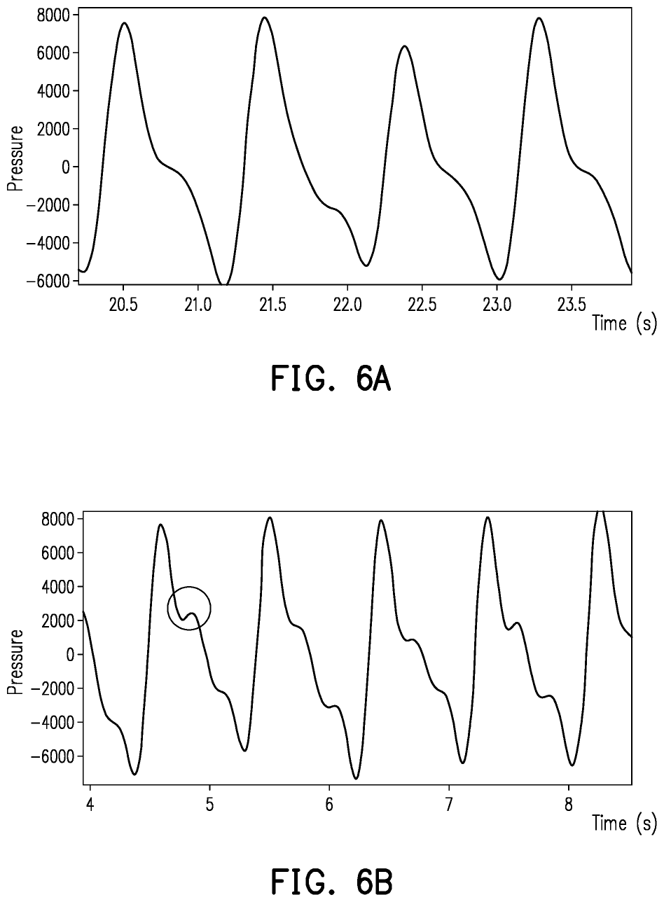

Please refer to FIG. 6A and FIG. 6B again. FIG. 6A depicts a pulse wave signal diagram corresponding to Curve I in FIG. 5A and FIG. 5B, and FIG. 6B depicts a pulse wave signal diagram corresponding to Curve II in FIG. 5A or Curve III in FIG. 5B. The vertical axis is relative pressure (arbitrary units). It can be seen that in the case where no elastomer is configured as shown in FIG. 6A, the pulse wave signal diagram cannot provide a tiny fluctuation (as circled in FIG. 6B) in each pulse wave like the pulse wave signal diagram obtained when the elastomers 101, 102, and 103 with appropriate thickness and appropriate hardness are configured in FIG. 6B.

Generally speaking, by arranging the elastomers 101, 102, and 103 with appropriate thickness and appropriate hardness between the sub air-bladders 201, 202, and 203 and the pressure sensors 101S, 102S, and 103S, the pressure from the sub air-bladders 201, 202, and 203 can be applied to each pulse point of the user more concentratedly, which increases the amplitude of the measured pulse wave, and can measure tiny fluctuations in each pulse wave more accurately to greatly improve the measurement sensitivity.

According to some embodiments of the invention, the hardness of each of the elastomers 101, 102, and 103 may be less than or equal to 50 HA. In some embodiments, the hardness of each of the elastomers 101, 102, and 103 is less than or equal to 10 HA. According to some embodiments of the invention, the thickness of each of the elastomers 101, 102, and 103 may be less than or equal to 5 mm. In some embodiments, the thickness of each of the elastomers 101, 102, and 103 is less than or equal to 2 mm.

In summary, by configuring the discrete sub air-bladders and elastomers, the physiological signal measurement apparatus provided by the embodiment of the invention can respectively apply appropriate pressures to pulse points with different depths, and each pressure sensor can be attached to the skin to accurately sense the pulse pressure of each pulse point, which greatly improves the measurement sensitivity and shortens the measurement time.

What is claimed is:

1. A physiological signal measurement apparatus, comprising:
a base seat;
an air-bladder device, disposed on the base seat and comprising a plurality of sub air-bladders;
a plurality of pressure sensors, respectively disposed on the plurality of sub air-bladders; and
a plurality of elastomers, respectively disposed between each of the plurality of sub air-bladders and a corresponding one of the plurality of pressure sensors.

2. The physiological signal measurement apparatus according to claim 1, wherein a hardness of the plurality of elastomers is less than or equal to 50 HA.

3. The physiological signal measurement apparatus according to claim 1, wherein a thickness of the plurality of elastomers is less than or equal to 5 mm.

4. The physiological signal measurement apparatus according to claim 1, wherein the plurality of sub air-bladders are communicated with each other.

5. The physiological signal measurement apparatus according to claim 1, wherein when the plurality of sub air-bladders are under a same inflation pressure, heights of the plurality of sub air-bladders relative to the base seat are different.

6. The physiological signal measurement apparatus according to claim 5, wherein the plurality of sub air-bladders comprise a first sub air-bladder, a second sub air-bladder, and a third sub air-bladder arranged on the base seat in sequence, when the first sub air-bladder to the third sub air-bladder are under the same inflation pressure, heights of the first sub air-bladder and the third sub air-bladder relative to the base seat are greater than a height of the second sub air-bladder relative to the base seat.

7. The physiological signal measurement apparatus according to claim 6, wherein the heights of the first sub air-bladder and the third sub air-bladder relative to the base seat are 1.1 to 1.5 times the height of the second sub air-bladder relative to the base seat.

8. The physiological signal measurement apparatus according to claim 1, wherein the base seat comprises a first wing and a second wing oppositely disposed and a connecting part connecting the first wing and the second wing, the first wing, the second wing, and the connecting part surround to form a containing part, and the air-bladder device, the plurality of pressure sensors, and the plurality of elastomers are disposed in the containing part.

9. The physiological signal measurement apparatus according to claim 8, wherein the first wing, the second wing, and the connecting part are integrally formed.

10. The physiological signal measurement apparatus according to claim 8, wherein the first wing and the second wing are disposed in parallel.
